(19) United States
(12) Patent Application Publication
(10) Pub. No.: US 2003/0078483 A1
(43) Pub. Date: Apr. 24, 2003

(54) Microelectrode supporting cell with excitable membrane

(76) Inventor: Thierry Herve, Claix (FR)

Correspondence Address: DUANE MORRIS LLP, 100 COLLEGE ROAD WEST, SUITE 100, PRINCETON, NJ, 08540-6604, US

(21) Appl. No.: 10/168,123

(22) PCT Filed: Dec. 14, 2000
(86) PCT No.: PCT/FR00/03543

(30) Foreign Application Priority Data
Dec. 14, 1999 (FR) 99/15772

(51) Int. Cl. A61B 5/04

(52) U.S. Cl. 600/372

(57) ABSTRACT

The invention concerns a microelectrode designed to support at least a cell with excitable membrane, comprising an insulating wafer (11) whereon are formed detecting electrodes. A cover above each electrode is enclosed with walls made of insulating material substantially perpendicular to the wafer for locking in position said at least one cell. Spacing means (26-29) enable said at least one cell to be at a specific distance from the corresponding electrode.

The present invention relates to a glycaemia detector, and more specifically to an electrode of very small dimensions likely to detect the electric activity of a cell or of a cluster of cells with an excitable membrane, for example, neurons, autonomous nodal heart cells or islets of Langerhans.

Such electrodes are known in the art and have in particular been described in U.S. Pat. No. 5,513,636 of Israeli Company CB-Carmel Biotechnology Ltd.

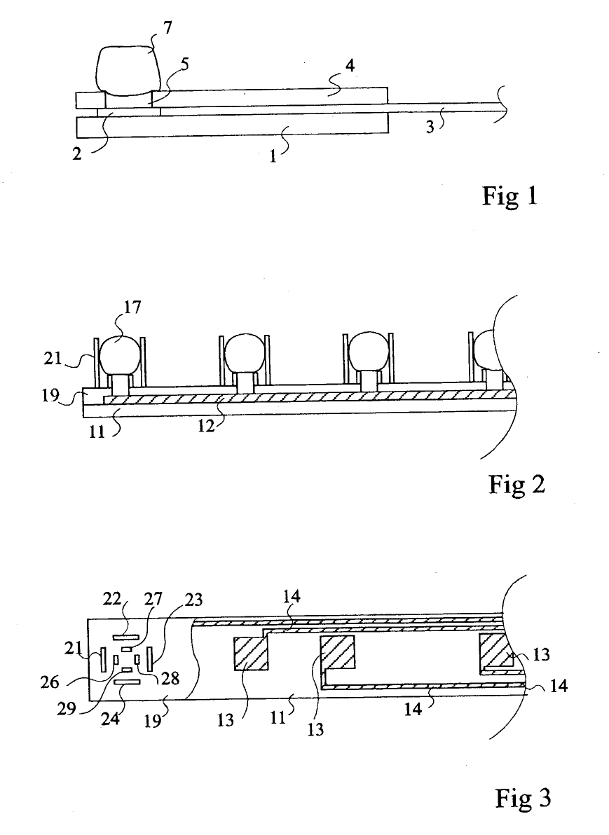

This patent describes an electrode of the type shown in FIG. 1 which includes a support plate 1 made of a rigid insulating material such as silicon or glass. On this plate is arranged an electrode formed of a conductive layer 2 connected to a wire 3. This conductive layer for example has a thickness smaller than 0.1 μm. Plate 1 is entirely covered with an insulating layer 4 provided with an opening 5 opposite to conductive layer 2. A cell, for example, an islet of Langerhans, is arranged on opening 5 to adhere to conductive layer 2 and to the walls of insulating layer 4. It is specified that “The exposed upper surface of the insulating layer on which the cells are to be at least partially grown (part of each cell grows on the conducting plate and part of the cell grows on the insulating layer) should be processed so that the cells grow on this layer and tend to strongly and sealingly adhere to it” (column 4, lines 14 to 18). It is also specified that “the strong adherence and seal between the cell and the insulating layer prevents the electrical signal from the cell from being attenuated by short circuiting between the cell or conducting plate and the medium surrounding the cell” (column 4, lines 22 to 26).

Thus, in this patent, it is provided for the cell or the islet of Langerhans to be very close to the corresponding electrode or in contact therewith. It is also provided for the portion of the surrounding fluid trapped between the cell and the electrode to be located in a chamber closed by the walls of opening 5, conductive layer 2, and cell 7.

Further, this document provides for several structures such as that illustrated in cross-section in FIG. 1 to be arranged in parallel on a same plate: openings 5 are side by side and wires 3 are parallel to one another, in a direction perpendicular to the cell alignment direction.

German patent application DE-A-19712309 aims at a microelectrode for cells of small diameter (10 μm) in which a contact between cells and electrodes is also wanted.

The applicant has acknowledged that, in practice, the electric signals detected by such electrodes were not optimal and has attempted to increase the electrode detectivity.

Further, the structure illustrated in FIG. 1 is intended to be incorporated in a capsule. In this incorporation, at least part of the islets of Langerhans appear in practice to fall off the opening above which they must be located or to aggregate together.

An object of the present invention is to overcome the disadvantages of this prior electrode and to provide an electrode with a higher detectivity threshold.

To achieve these objects, the present invention provides a microelectrode intended to support at least one cell with an excitable membrane, including an insulating plate provided with openings, each opening emerging on a detection electrode and being surrounded with walls made of insulating material, substantially perpendicular to the plate to block in position said at least one cell, spacing means being provided for maintaining said at least one cell at a determined distance from the corresponding electrode.

According to an embodiment of the present invention, the spacing means are walls made of an insulating material, substantially perpendicular to the plate.

According to an embodiment of the present invention, the walls are formed from a multiple-layer structure of insulating material.

According to an embodiment of the present invention, the multiple-layer structure is formed by successive steps of spin-on deposition and of anneal of a polyimide.

According to an embodiment of the present invention, said opening is closed by an insulating plate on which the electrode is laid.

According to an embodiment of the present invention, said opening is a through opening and the detection electrode is arranged on the surface opposite to that supporting said at least one cell, at the periphery of the opening.

According to an embodiment of the present invention, said at least one cell with an excitable membrane is an islet of Langerhans.

According to an embodiment of the present invention, several microelectrodes are stacked, the walls of insulating material being used as spacers between two superposed electrodes.

The foregoing objects, features and advantages of the present invention, will be discussed in detail in the following non-limiting description of specific embodiments in connection with the accompanying drawings.

FIG. 1 is a partial simplified cross-section view of an electrode according to prior art;

FIG. 2 is a partial simplified cross-section view of an electrode according to an embodiment of the present invention;

FIG. 3 is a simplified top view of the electrode of FIG. 2;

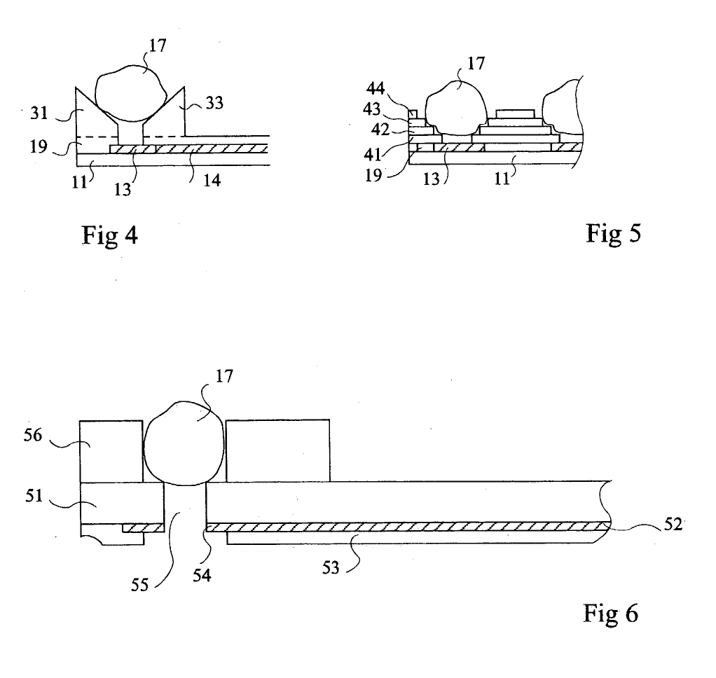

FIG. 4 is a partial cross-section view illustrating another embodiment of the present invention;

FIG. 5 is a partial cross-section view illustrating another embodiment of the present invention; and

FIG. 6 is a partial cross-section view illustrating another embodiment of the present invention.

In the various drawings, the thicknesses of the various layers and supports are not to scale and may be conventionally chosen by those skilled in the art.

FIG. 2 is a cross-section view partially and schematically showing a first embodiment of the present invention. A thin layer of a conductor, currently a metal, for example, gold, properly etched to define conductive plates forming electrodes and connection tracks, is deposited, for example, by evaporation, on an insulating support plate 11, for example, a film of small thickness (a few μm). In the right-hand portion of the partially cut-away top view of FIG. 3, an example of conductive plates 13 and of connection tracks 14 can be seen. An insulating layer 19 is then deposited, for example, by spin-on deposition. This technique enables depositing layers having thicknesses from a few μm to a few tens of μm according to the spinning rate. A layer of polyimide mixed with a solvent may for example be deposited, then annealed. Insulating layer 19 is provided with openings above the locations of the detection electrodes, corresponding to sites where islets of Langerhans 17 are to be placed.

According to an aspect of the present invention, each deposition site of cells, for example, islets of Langerhans, is surrounded with lateral blocking means, for example, four vertical walls 21-24. The spacing between these walls is chosen to be greater than or equal to the average value of the diameter of the considered cells, this average value being generally on the order of 100 μm for an islet of Langerhans. Walls 21-24 may be formed by thick layer deposition, or preferably by successive depositions of an insulator and etching. They will for example have a height slightly smaller than the diameter of an islet of Langerhans. The etching may for example be performed by reactive ionic etch (RIE) in the presence of O₂ or of CHF₃ or by excimere laser, which enables etching in a substantially vertical fashion layers having thicknesses on the order of 100 μm. As can be seen in FIG. 3, the walls are “open-worked”, that is, they do not form a continuous contour. In the example shown, they do not join at the level of the corners of the square that they define. This is intended to enable nutriments (the matter in which the device will be placed) to reach all the cells of the islet of Langerhans.

According to a second aspect of the present invention; spacing means for bringing the low portion of each islet of Langerhans to a substantially constant height with respect to the corresponding detection electrode are provided. Indeed, according to this aspect of the present invention, it has been determined that the voltage detected by an electrode is significantly increased when the distance between the low portion of an islet of Langerhans and the corresponding detection electrode has a determined value, which is small but not zero. A distance on the order of half the diameter of the islet of Langerhans may be chosen, for example, a distance ranging between 0.2 and 0.7 times the diameter of the islet of Langerhans, although other values are possible. For other cells or groups of cells, an optimal distance may similarly be chosen.

In the case of FIGS. 2 and 3, the spacing means are formed of small walls 26-29, on the tops of which the islet of Langerhans bears.

The materials constitutive of the components of the electrode illustrated in FIGS. 2 and 3 will be chosen to enable simple manufacturing and be biocompatible. For example, the various insulating materials will be polyimides such as polyimide PI 2611 of Dupont de Nemours and the conductive materials will be gold layers.

As shown in FIG. 3, on a same electrode plate, several sites arranged one before the other lengthwise on the plate will be provided. Thus leads to forming a set of electrodes of small size and particularly simple to implant in a capsule intended to be placed in a patient's body. Although, to make the representation clearer, the cells are shown as being spaced apart by a distance greater than their diameter, it may be provided for the walls to be arranged so that two close cells are very close to each other, the walls enabling avoiding for two neighboring cells to cling on to each other.

According to an aspect of the present invention, separation walls higher than the diameter of a cell may be provided and several microelectrodes may be stacked, the separating walls being used as spacers between two superposed microelectrodes.

FIGS. 4 to 6 show various alternative embodiments of a detection electrode site.

In the embodiments of FIGS. 4 and 5, the lateral walls and the spacing walls are formed by one and the same structure.

In the case of FIG. 4, walls 21-24 are replaced with walls 31-34 (only walls 31 and 33 are visible in the cross-section view). Wall 31 plays the role of lateral blocking wall 21 and of spacing wall 26. Similarly, wall 33 plays the role of lateral blocking wall 23 and of spacing wall 28. For this purpose, walls 31 and 33 are relatively wide and have upper surfaces slanted towards the inside.

FIG. 5 shows a structure which is generally identical to that of FIG. 4 but which results from a deposition of multiple layers which are successively appropriately etched to form the slanted planes stepwise. Thus, a succession of layers 41, 42, 43, 44, . . . for example, some ten successive layers each having a thickness on the order of from a few μm to a few tens of μm are deposited, each layer of a first material being covered with a very thin layer of a second material behaving as an etch stop. Successive etchings according to narrower and narrower concentric windows are then performed to obtain the shown step structures. Conversely, an etching may be performed after each deposition, the successive layers being etched to form openings wider than previous openings, and centered thereon.

FIG. 6 shows another embodiment of the present invention which essentially differs from the preceding embodiments in that the conductive layer forming the detection electrode is not placed at the back of an opening corresponding to a blind hole, but on the surface opposite to the islet of Langerhans of a through opening. The embodiment of FIG. 6 starts with a relatively thick but flexible support 51 having its lower surface coated with a metallization 52. A protection insulating layer 53 is deposited on this metallization. Layer 53 is locally removed to expose a detection electrode area 54 at the level of each detection site. At the level of each of these sites, support plate 51 is opened by a through hole 55 so that detection electrode 54 forms a ring peripheral to the opening on the lower surface side of plate 51. In this embodiment, the thickness of plate 51 forms the spacing means setting the distance between the lower part of islet of Langerhans 17 and detection electrode 54. On its upper surface side is deposited a thick layer of insulating material 56 including openings wider than openings 55 at the level of each detection site. Layer 56 and the corresponding openings correspond to the previously-described lateral blocking means.

In the embodiment of FIG. 6, it should be noted that, due to the fact that opening 55 is a through opening, nutriments can arrive on the lower side of the islet of Langerhans through this opening. Thus, it is not necessary for wall 56 laterally blocking the islet of Langerhans to be open-worked. A circular opening in a thick layer may for example be provided.

Of course, the various embodiments of the present invention may be combined by those skilled in the art.

1. A microelectrode intended to support at least one cell with an excitable membrane immerged in a fluid, including an insulating plate (11, 51) provided with openings, each opening emerging on a detection electrode (13, 54) and being surrounded with walls made of insulating material, substantially perpendicular to the plate to block in position said at least one cell, characterized in that it further includes spacing means (26-29) for maintaining said at least one cell at a determined distance from the corresponding electrode, said distance being occupied by said fluid.

2. The microelectrode of claim 1, characterized in that the spacing means are walls made of an insulating material, substantially perpendicular to the plate.

3. The microelectrode of claim 1, characterized in that the walls are formed from a multiple-layer structure of insulating material.

4. The microelectrode of claim 3, characterized in that the multiple-layer structure is formed by successive steps of spin-on deposition and of anneal of a polyimide.

5. The microelectrode of claim 1, wherein said opening is closed by an insulating plate on which the electrode is laid.

6. The microelectrode of claim 1, characterized in that said opening is a through opening and the detection electrode is arranged on the surface opposite to that supporting said at least one cell, at the periphery of the opening.

7. The microelectrode of claim 1, characterized in that said at least one cell with an excitable membrane is an islet of Langerhans.

8. A stack of the microelectrodes of any of claims 1 to 7, in which the walls of insulating material are used as spacers between two superposed microelectrodes.
